(19) United States
(12) Patent Application Publication
(10) Pub. No.: US 2022/0238188 A1
(43) Pub. Date: Jul. 28, 2022

(54) METHOD FOR DETERMINING RESPONSIVENESS TO AN EPITOPE

(71) Applicants: UNIVERSITEIT ANTWERPEN, Antwerpen (BE); UNIVERSITAIR ZIEKENHUIS ANTWERPEN, Edegem (BE)

(72) Inventors: Pieter Meysman, Leuven (BE); Kris Laukens, Wilrjk (BE); Benson Ogunjimi, Meise (BE)

(73) Assignees: UNIVERSITEIT ANTWERPEN, Antwerpen (BE); UNIVERSITAIR ZIEKENHUIS ANTWERPEN, Edegem (BE)

(21) Appl. No.: 17/421,420

(22) PCT Filed: Feb. 28, 2020
(86) PCT No.: PCT/EP2020/055224

§ 371 Date: Jul. 8, 2021

(30) Foreign Application Priority Data
Feb. 28, 2019 (EP) 19159931.5

(51) Int. Cl. G16B 40/20; G16B 20/30; G16B 5/00

(52) U.S. Cl. CPC G16B 40/20; G16B 5/00; G16B 20/30

(57) ABSTRACT

Method (100) for determining an immune responsiveness to a query epitope (126) comprising: receiving sequence data (122) comprising TCR sequences of at least a part of a TCR repertoire of a subject; selecting a predictive model (160) generated or trained using a dataset comprising TCR sequences known to bind specifically to a model epitope, said predictive model selected according to a sequence match between the model epitope and query epitope; querying (130) the selected predictive model (160) with the sequence data (122); determining (140) from outputs of the selected predictive model (160) a Responsiveness Score indicative of the immune responsiveness. The immune responsiveness can be used to predict and optimal vaccine composition and/or evaluate efficacy of a vaccine in a subject or population.

### CROSS-REFERENCE TO RELATED APPLICATIONS

This application is a U.S. national stage entry under 35 U.S.C. § 371 of PCT International Patent Application NO. PCT/EP2020/055224, filed Feb. 28, 2020, which claims priority to European Patent Application NO. 19159931.5, filed Feb. 28, 2019, the contents of each of which are incorporated herein by reference in their entirety.

### FIELD OF THE INVENTION

The present invention is in a field of immunology and medicine.

### BACKGROUND TO THE INVENTION

Presently, there are few methods or assays existing to predict how a subject will respond to a vaccine given the vaccine content and the individual characteristics. Methods and assays for predicting a beneficial, preferably optimal, response (such as antibody response) would be useful for any type of vaccination. This kind of predictive tools would have several benefits. For example, on an individual level it would be made possible to predict whether a certain vaccine would be able to elicit an immunological and/or clinical response. For example, on a population level it would be made possible to assess whether a “successful” vaccine would likely be successful in another genetic population.

Xu Jin et al “Immunological Recognition by Artificial Neural Networks”, Journal of the Korean Physical Society, Korean Physical Society, vol. 73, no. 12, 29, pages 1908-1917 describes the use of a single trained model to predict binding of single epitopes to a TCR sequence. WO 2018/183980 describes the use of a trained model to predict immunogenic T-cell neo-epitopes from somatic variants (disease-associated mutations).

### SUMMARY

Provided is a method (100) for determining an immune responsiveness to a query epitope (126) comprising:
receiving sequence data (122) comprising TCR sequences of at least a part of a TCR repertoire of a subject,
selecting a predictive model (160) generated or trained using a dataset comprising TCR sequences known to bind specifically to a model epitope, said predictive model selected according to a sequence match between the model epitope and query epitope,
querying (130) the selected predictive model (160) with the sequence data (122),
determining (140) from outputs of the selected predictive model (160) a Responsiveness Score indicative of the immune responsiveness of the subject to the query epitope (126).

Provided is a method (100) for determining an immune responsiveness to a query epitope (126) comprising:
receiving sequence data (122) comprising TCR sequences of at least a part of a TCR repertoire of a subject,
selecting a predictive model (160) from a plurality of a predictive models (PM_{ME-A}, PM_{ME-B}, PM_{ME-C}, . . . ), wherein each predictive model (PM_{ME-A}, PM_{ME-B}, PM_{ME-C}, . . . ) in the plurality has been generated or trained using a dataset comprising a plurality of TCR sequences known to bind specifically to one model epitope (ME-A, ME-B, ME-C, . . . ), said predictive model (160) selected according to a sequence identity match between the model epitope (ME-A, ME-B, ME-C, . . . ) and query epitope (126),
querying (130) the selected predictive model (160) with the sequence data (122),
determining (140) from outputs of the selected predictive model (160) a Responsiveness Score indicative of the immune responsiveness of the subject to the query epitope (126).

The method may be used to predict an optimum vaccine. The vaccine may comprise at least one query epitope (126). At least one query epitope (126) may be identified from the vaccine that sequence matches a model epitope in the model database and the predictive model is linked to the matched model epitope in the model database.

Also provided is a method (100) for predicting for a subject an optimal vaccine composition from a set (120) of query epitopes (126), by determining an immune responsiveness of the subject to each query epitope (126) in the set (120) comprising:

receiving sequence data (122) comprising TCR sequences of at least a part of a TCR repertoire of the subject prior to vaccine administration,
selecting, for each query epitope (126) in the set of query epitopes (120), a predictive model (160) from a plurality of predictive models (PM_{ME-A}, PM_{ME-B}, PM_{ME-C}, . . . ),
   wherein each predictive model (PM_{ME-A}, PM_{ME-B}, PM_{ME-C}, . . . ) in the plurality of predictive models has been generated or trained using a dataset comprising a plurality of TCR sequences known to bind specifically to one model epitope (ME-A, ME-B, ME-C, . . . ),
   said predictive model (160) selected according to a sequence identity match between the model epitope (ME-A, ME-B, ME-C, . . . ) and query epitope (126),
querying (130) each selected predictive model (160) with the sequence data (122),
determining (140) from outputs of the selected predictive model (160) a Responsiveness Score for each query epitope (126) in the set (120) indicative of the immune responsiveness of the subject to the query epitope (126),
predicting the optimal vaccine composition for the subject from the Responsiveness Scores.

Also provided is a method (100) for predicting for a population an optimal vaccine composition from a set (120) of query epitopes (126), by determining an immune responsiveness of each subject of a set of reference subjects to each query epitope (126) in the set (120) comprising:
receiving sequence data (122) comprising TCR sequences of at least a part of a TCR repertoire of each subject in the set of reference subjects prior to vaccine administration,
selecting, for each query epitope (126) in the set of query epitopes (120), a predictive model (160) from a plurality of predictive models (PM_{ME-A}, PM_{ME-B}, PM_{ME-C}, . . . ),
   wherein each predictive model (PM_{ME-A}, PM_{ME-B}, PM_{ME-C}, . . . ) in the plurality of predictive models has been generated or trained using a dataset comprising a plurality of TCR sequences known to bind specifically to a model epitope (ME-A, ME-B, ME-C, . . . ),
   said predictive model (160) selected according to a sequence identity match between the model epitope (ME-A, ME-B, ME-C, . . . ) and query epitope (126),
querying (130) each selected predictive model (160) with the sequence data (122),
determining (140) from outputs of the selected predictive model (160) a Responsiveness Score for each query epitope (126) in the set (120) indicative of the immune responsiveness of the subject to the query epitope (126),
predicting the optimal vaccine composition for the population from the Responsiveness Scores for the set of reference subjects.

Also provided is a method for evaluating efficacy of a vaccine (170) in a subject by determining an immune responsiveness to at least one query epitope (126) identified from the vaccine (170) comprising:
receiving sequence data (122) comprising TCR sequences of at least a part of a TCR repertoire of a subject prior to vaccine administration,
selecting, for each query epitope (126), a predictive model (160) from a plurality of predictive models (PM_{ME-A}, PM_{ME-B}, PM ME-C, . . . ),
   wherein each predictive model (PM_{ME-A}, PM_{ME-B}, PM_{ME-C}, . . . ) in the plurality of predictive models has been generated or trained using a dataset comprising a plurality of TCR sequences known to bind specifically to a model epitope (ME-A, ME-B, ME-C, . . . ),
   said predictive model (160) selected according to a sequence identity match between the model epitope (ME-A, ME-B, ME-C, . . . ) and query epitope (126),
querying (130) each selected predictive model (160) with the sequence data (122),
determining (140) from outputs of the selected predictive model (160) a Responsiveness Score for each query epitope (126) indicative of the immune responsiveness of the subject to the query epitope (126),
evaluating (140) from the Responsiveness Score of each query epitope (126) the efficacy of the vaccine for the subject.

Also provided is a method for evaluating efficacy of a vaccine (170) in a population by determining an immune responsiveness to at least one query epitope (126) identified from the vaccine (170) comprising:
receiving sequence data (122) comprising TCR sequences of at least a part of a TCR repertoire of each subject of a set of reference subjects prior to vaccine administration,
selecting, for each query epitope (126) in the set of query epitopes (120), a predictive model (160) from a plurality of predictive models (PM_{ME-A}, PM_{ME-B}, PM_{ME-C},
   wherein each predictive model (PM_{ME-A}, PM_{ME-B}, PM_{ME-C}, . . . ) in the plurality of predictive models has been generated or trained using a dataset comprising a plurality of TCR sequences known to bind specifically to a model epitope (ME-A, ME-B, ME-C, . . . ),
   said predictive model (160) selected according to a sequence identity match between the model epitope (ME-A, ME-B, ME-C, . . . ) and query epitope (126),
querying (130) each selected predictive model (160) with the sequence data (122),
determining (140) from outputs of the selected predictive model (160) a Responsiveness Score for each query epitope (126) indicative of the immune responsiveness of the subject to the query epitope (126),
determining (140) from the Responsiveness Scores the efficacy of the vaccine for the set of reference subjects.

The predictive model (160) may be selected from a model database (150) comprising a plurality of predictive models (PM_{ME-A}, PM_{ME-B}, PM_{ME-C}, . . . ) each trained using a different dataset and each linked to the model epitope (ME-A, ME-B, ME-C, . . . ) of the dataset.

The predictive model may be a machine learning model trained using the training dataset.

The sequence match between the model epitope and query epitope may be determined by sequence identity.

The total quantity of TCR sequences in the sequence data (122) may be a fraction of the total number of available TCR sequences in the repertoire of the subject.

The sequence data (122) may be comprise TCR sequences that are only antigen-experienced TCR sequences from the TCR repertoire of the subject

Further provided is a method for obtaining at least one Responsiveness Score for a vaccine to prior administration to a subject comprising the method described herein, wherein
the sequence data comprises TCR sequences of part of the TCR repertoire of the subject prior to vaccine administration,
at least one query epitope is identified from the vaccine that sequence matches a model epitope in the model database and the predictive model is linked to the matched model epitope in the model database,
a Responsiveness Score is obtained for each query epitope of the vaccine for the subject.

Further provided is a method for obtaining at least one Responsiveness Score for a vaccine in a population to prior administration comprising the method described herein, wherein
the sequence data comprises TCR sequences of part of the TCR repertoire of each subject of a set of reference subjects representative of the population prior to vaccine administration,
at least one query epitope is identified from the vaccine that sequence matches a model epitope in the model database and the predictive model is linked to the matched model epitope in the model database,
a Responsiveness Score is obtained for each query epitope of the vaccine for each reference subject.

Further provided is a method for evaluating efficacy of a vaccine (170) in a subject to prior administration comprising the method described herein, wherein
the sequence data (122) comprises TCR sequences of part of the TCR repertoire of the subject prior to vaccine administration,
at least one query epitope (126) is identified from the vaccine (170) that sequence matches a model epitope in the model database (150) and the predictive model (160) used in the querying step (130) is linked to the matched model epitope in the model database (150),
the efficacy of the vaccine for the subject is determined (140) from the Responsiveness Score of each query epitope.

Further provided is a method for evaluating efficacy of a vaccine (170) in a population to prior administration comprising the method described herein, wherein
the sequence data (122) comprises TCR sequences of part of the TCR repertoire of each subject of a set of reference subjects prior to vaccine administration,
at least one query epitope (160) is identified from the vaccine that sequence matches a model epitope in the model database (150) and the predictive model (used in the querying step (130)) is linked to the matched model epitope in the model database (150),
the efficacy of the vaccine for the set of reference subjects is determined (140) from the Responsiveness Scores for the set of reference subjects, and the set of reference subject is indicative for the population.

Further provided is a method for predicting for a subject an optimal vaccine from a set of query epitopes, comprising the method described herein, wherein:
the sequence data comprises TCR sequences of part of the TCR repertoire of each subject of a set of the subject prior to vaccine administration,
each query epitope is searched against the model database to find a match to a model epitope and the predictive model (used in the querying step) is linked to the matched model epitope in the model database,
a Responsiveness Score is obtained for each query epitope,
the optimal vaccine for the subject is determined from the Responsiveness Score of each query epitope.

Further provided is a method for predicting for a population an optimal vaccine from a set of query epitopes, comprising the method described herein, wherein:
the sequence data comprises TCR sequences of part of the TCR repertoire of each subject of a set of reference subjects prior to vaccine administration,
each query epitope is searched against the model database to find a match to a model epitope and the predictive model (used in the querying step) is linked to the matched model epitope in the model database,
a set of Responsiveness Scores is obtained for each subject of the set, the set containing a Responsiveness Score for each query epitope,
the optimal vaccine for the population is determined from the Responsiveness Score of each query epitope for the set of reference subjects.

The vaccine may comprise one or more of:
at least one amino acid chain (protein, polypeptide, peptide)
at least one nucleic acid (double or single stranded RNA, DNA; DNA-RNA hybrid)
at least one immune system cell (e.g. antigen presenting cell, T-cell, B-cell, macrophage),
at least one an infectious agent (e.g. prokaryotic cell (bacteria),
at least one eukaryotic cell (yeast),
at least one virus,
at least one prion,
   and the at least one query epitope is present in the substance or vaccine as part of an amino acid sequence of the same length as the query epitope or longer than the query epitope, and/or as nucleic acid encoding said amino acid sequence of the same length as the query epitope or longer than the query epitope.

### FIGURE LEGENDS

FIGS. 1 to 3 show exemplary flow charts of the method described herein.

FIG. 4 depicts an ROC curve.

### DETAILED DESCRIPTION OF INVENTION

Before the present system and method of the invention are described, it is to be understood that this invention is not limited to particular systems and methods or combinations described, since such systems and methods and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any or etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the present description of the invention, reference is made to the accompanying drawings that form a part hereof, and in which are shown by way of illustration only of specific embodiments in which the invention may be practiced. Parenthesized or emboldened reference numerals affixed to respective elements merely exemplify the elements by way of example, with which it is not intended to limit the respective elements. It is to be understood that other embodiments may be utilised and structural or logical changes may be made without departing from the scope of the present invention. The following detailed description, therefore, is not to be taken in a limiting sense, and the scope of the present invention is defined by the appended claims.

Described herein is a method for determining an immune responsiveness to a query epitope. The method comprises receiving sequence data comprising T-cell receptor (TCR) sequences of at least a part of a TCR repertoire of a subject. A predictive model is selected, the predictive model having been trained or generated using a dataset comprising TCR sequences known to bind specifically to a model epitope. The predictive model may be selected according to a sequence match between the query epitope and the model epitope.

The sequence data is used to query the selected predictive model. The model may be queried for each TCR sequence present in the subject sequence data. From outputs of the selected predictive model, a Responsiveness Score is determined indicative of the immune responsiveness of the subject to the query epitope.

The predictive model may be selected according to a sequence match between the query epitope and the model epitope. The predictive model may be selected from a database comprising a plurality of predictive model each linked with a corresponding model epitope.

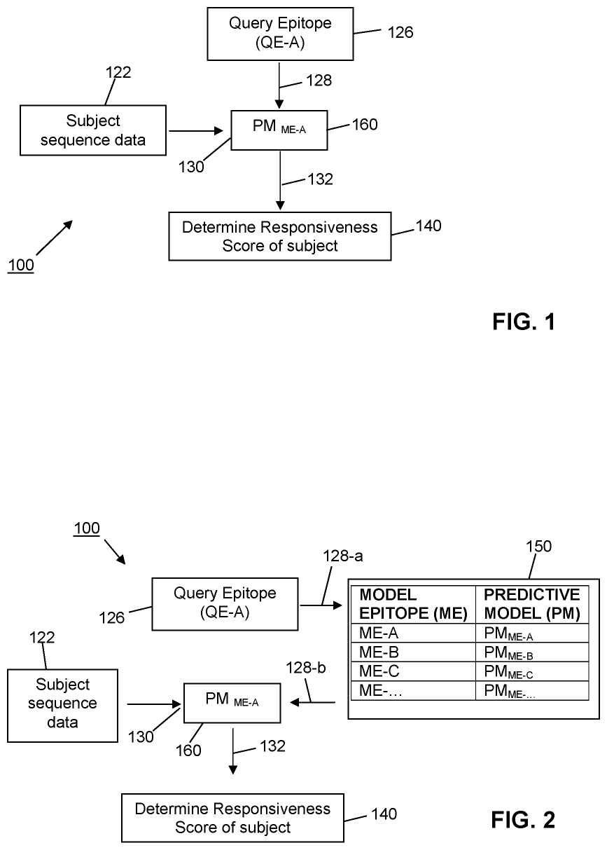

FIG. 1 shows an exemplary flow chart of the method (100) described herein. Subject sequence data (122) is received. Based on the query epitope (QE-A) (126) a predictive model (160) (PM_{ME-A}) is selected (128), the predictive model (160) (PM_{ME-A}) having been trained or generated using a dataset comprising TCR sequences known to bind specifically to a model epitope (ME-A). The selected predictive model (160) (PM_{ME-A}) is queried (130) with the sequence data (122). From an output (132) of the selected predictive model (160) the Responsiveness Score of the subject is determined (140).

FIG. 2 shows a further exemplary flow chart of the method (100) described herein. Subject sequence data (122) is received. Based on the query epitope (126) a predictive model (160) (PM_{ME-A}) is selected (128-a, 180-b) from a model database (150). The model database (150) comprises a plurality of predictive models (PM_{ME-A}, PM_{ME-B}, PM_{ME-C}) trained or generated using a dataset comprising TCR sequences known to bind specifically to a model epitope (ME-A, ME-B, ME-C), each predictive model (PM_{ME-A}, PM_{ME-B}, PM_{ME-C}, . . . ) linked to a model epitope (ME-A, ME-B, ME-C, . . . ). The selected predictive model (160) (PM_{ME-A}) is queried (130) with the sequence data (122). From an output (132) of the selected predictive model (160) the Responsiveness Score of the subject is determined (140).

The subject refers to a person whose responsiveness is to be determined. The subject is typically a mammal, typically a human. The method receives data related to a part of a TCR repertoire of the subject. In some circumstances, the TCR repertoires of several subjects (set of reference subjects) are used to determine responsiveness for a population.

Typically a query epitope is an amino acid sequence. It is understood that a query epitope may be an amino acid sequence translated from a nucleic acid sequence. Typically a query epitope is an epitopic stretch of amino acids that is 7 to 33 amino acids in length. The query epitope represents a putative minimum amino acid sequence that can be recognised by an immune system component (e.g. by a T-cell receptor). The query epitope is preferably a linear epitope.

The predictive model may be selected from a model database of predictive models. Each predictive model has been trained or generated using a dataset comprising TCR sequences known to bind specifically to at least one (preferably one) model epitope (sequence). The model database comprises a plurality of predictive models each trained using a different dataset. Each predictive model in the database is linked to the at least one (preferably one) model epitope. The database may be indexed at least according to the model epitope, and a search in the database of the model epitope will retrieve the matching predictive model or models. Where a match is found, the selected model or models are used in the method, namely in the querying step.

The model database may be any type of database, typically a computer-implemented database stored on a computer storage medium. The model database may be accessible across a network, for instance, over an intranet or the Internet (cloud).

The match between query epitope and model epitope may be based on a sequence identity and/or sequence similarity score between the respective epitopes. As understood herein, sequence identity is the degree to which a pair of amino acid sequences is invariant. Sequence identity is, for instance, expressed as a percentage that represents the fraction of invariant characters between the respective pair of epitope amino sequences. As understood herein, sequence similarity is the degree to which a pair of amino sequences is physicochemically similar or conserved. Techniques for measurement of sequence similarity include, for instance, Needleman and Wunsch (JMB, Volume 48, Issue 3, 28 Mar. 1970, Pages 443-453), and make use of substitution matrices, for instance the Dayhof PAM matrix (Dayhoff, M. O., R. M. Schwartz, and B. C. Orcutt. “22 a model of evolutionary change in proteins.” Atlas of protein sequence and structure (1978): 345-352), or the BLOSUM matrix (Henikoff and Henikoff, PNAS Nov. 15, 1992 89 (22) 10915-10919).

Preferably, a sequence match between query epitope and model epitope arises when they have at least an 80%, preferably at least a 90% sequence identity. Preferably, a sequence match between query epitope and model epitope arises when they are identical or differ only by a 1 or 2 amino acid substitution, deletion or insertion. Because the query epitope may not be an identical match to the model epitope, the query epitope may be 0, 1 or 2 amino acids longer or shorter.

The query epitope and model epitope are preferably linear epitopes.

The degree of similarity or matching between the query epitope and model epitope may be used as a weighting factor in determining the Responsiveness Score.

The method described herein may be used to determine a Responsiveness Score for at least one query epitope contained in a substance or vaccine comprising at least one of:
at least one amino acid chain (protein, polypeptide);
at least one nucleic acid (double or single stranded RNA, DNA; DNA-RNA hybrid);
at least one immune system cell (e.g. antigen presenting cell, T-cell, B-cell, macrophage);
at least one an infectious agent (e.g. prokaryotic cell (bacteria);
at least one eukaryotic cell (yeast);
at least one virus;
at least one prion.

The method described herein may be used to determine an efficacy of a vaccine and/or other predictive measure for a substance or vaccine containing at least one query epitope, the substance or vaccine comprising at least one of:
at least one amino acid chain (protein, polypeptide);
at least one nucleic acid (double or single stranded RNA, DNA; DNA-RNA hybrid);
at least one immune system cell (e.g. antigen presenting cell, T-cell, B-cell, macrophage);
at least one an infectious agent (e.g. prokaryotic cell (bacteria);
at least one eukaryotic cell (yeast);
at least one virus;
at least one prion.

The at least one query epitope may be present in the substance or vaccine as part of an amino acid sequence of the same length as the query epitope or longer than the query epitope, and/or as nucleic acid encoding said amino acid sequence of the same length as the query epitope or longer than the query epitope.

The substance or vaccine may comprise a polypeptide containing the at least one query epitope, wherein the amino acid sequence of the polypeptide is the same length as the at least one query epitope or longer than the at least one query epitope; the substance or vaccine may comprise a nucleic acid encoding said polypeptide.

The substance or vaccine may comprise a protein containing the at least one epitope, wherein the amino acid sequence of the protein is longer than the query epitope or longer than the query epitope; the substance or vaccine may comprise a nucleic acid encoding said protein.

The query epitopes of the substance or vaccine may be from a same target protein or different target proteins.

The method may comprise further steps of identifying, from the substance or vaccine, one or more query epitopes. The query epitope may be identified by searching a substance or vaccine amino acid sequence(s) for the presence of a model epitope sequence from the model database. Where the substance or vaccine contains nucleic acid, the nucleic acid is first translated into corresponding amino acid sequence. Typically a model epitope sequence from the model database is moved residue-by-reside along a substance or vaccine amino acid sequence, and where there is a match that substance or vaccine amino acid sequence is assigned a query epitope for the method.

The match between query epitope and model epitope may be based on a sequence identity and/or sequence similarity score between the respective epitopes. As understood herein, sequence identity is the degree to which a pair of amino acid sequences is invariant. Sequence identity is, for instance, expressed as a percentage that represents the fraction of invariant characters between the respective pair of epitope amino sequences. As understood herein, sequence similarity is the degree to which a pair of amino sequences is physicochemically similar or conserved. Techniques for measurement of sequence similarity include, for instance, Needleman and Wunsch (JMB, Volume 48, Issue 3, 28 Mar. 1970, Pages 443-453), and make use of substitution matrices, for instance the Dayhof PAM matrix (Dayhoff, M. O., R. M. Schwartz, and B. C. Orcutt. “22 a model of evolutionary change in proteins.” Atlas of protein sequence and structure (1978): 345-352), or the BLOSUM matrix (Henikoff and Henikoff, PNAS Nov. 15, 1992 89 (22) 10915-10919).

Preferably, a sequence match between model epitope and putative epitope in the substance or vaccine or amino acid sequence arises when they have at least an 80%, preferably at least a 90% sequence identity. Preferably, a sequence match between query epitope and model epitope arises when they are identical or differ only by a 1 or 2 amino acid substitution, deletion or insertion. Because the query epitope may not be an identical match to the model epitope, the query epitope may be 0, 1 or 2 amino acids longer or shorter.

Those matching epitopes are extracted from the substance or vaccine amino acid sequences. Each matching model epitope is associated with a predictive model in the database; the associated predictive model is used in the querying step of the method to determine a Responsiveness Score for that epitope in the subject.

FIG. 3 shows a further exemplary flow chart of the method (100) described herein further comprising steps of identifying, from the substance or vaccine (170), one or more query epitopes (126). Substance or vaccine sequence data (170) is inputted (124) into an identification protocol wherein each model epitope from the model database (150) (ME-A, ME-B, ME-C) is used to search against the substance or vaccine sequence data (170) for sequence matches. The model database (150) comprises a plurality of predictive models (PM_{ME-A}, PM_{ME-B}, PM_{ME-C}) trained or generated using a dataset comprising TCR sequences known to bind specifically to a model epitope (ME-A, ME-B, ME-C), each predictive model (PM_{ME-A}, PM_{ME-B}, PM_{ME-C}, . . . ) linked to a model epitope (ME-A, ME-B, ME-C, . . . ). Outputted (125) are query epitopes (126) (QE-A) of the substance or vaccine sequence data (170) that match the model epitopes (ME-A) in the model database (150). The outputted matching query epitopes (126) (QE-A) are linked in the database to predictive models (PM_{ME-A}); they are retrieved (128-c, -d) and used for the querying step (130). The selected predictive model (160) (PM_{ME-A}) is queried (130) with the sequence data (122). The method is repeated for all query epitopes identified. From the outputs (132) of the selected predictive models (160) the responsiveness score of the subject for the substance or vaccine is determined (140).

All or a set of model epitope sequences from the model database may be searched against the substance or vaccine amino acid sequences. Where a plurality of matching query epitopes is found in the substance or vaccine amino acid sequences, each associated predictive model is used in successive cycles of the method (namely in the querying step), until all the matching query epitopes have been exhausted.

The substance or vaccine amino acid sequences may be known, for instance, from a knowledge of the composition of the substance, for instance, containing an expression product of a vector. Alternatively, the substance amino acid sequences may be identified by sequencing the substance amino acid (e.g. nucleic acid or amino acid).

Sequence data refers to plurality of TCR amino acid sequences or of TCR nucleic acid sequences that are translated into amino acid sequences. The sequence data may comprise more than 10⁵ TCR separate sequences of the subject, preferably up to 10⁷ TCR separate sequences of the subject. The sequence data may contain redundancies, for instance, owing to the presence of multiple T-cells of a specific clonotype TCR sequence). The sequence data may contain no redundancies. The total quantity of TCR sequences in the sequence data may be a fraction of the total number of available TCR sequences in the repertoire of the subject.

In some cases sequence data may comprise TCR sequences of a T-cell clonotypes with a read count greater than 10, wherein the read count of a TCR sequence of T-cell clonotype is determined from the number TCR sequences read (i.e. sequenced) matching the sequences of the T-cell clonotype.

The TCR repertoire of a subject refers to a T-cell receptor sequence repertoire of the subject. A part of the TCR repertoire of a subject is sequenced.

A TCR sequence may be determined by sequencing gDNA or cDNA derived from the T-cell. The TCR sequence of a T-cell receptor may comprises one or more of TCR beta-chain, TCR alpha-chain, or both, or part thereof. The TCR sequence of a T-cell receptor may comprises one or more of a complementary determining region of a TCR beta-chain, TCR alpha-chain, or both, or part thereof. The TCR sequence of a T-cell receptor may comprises one or more of beta-chain CDR3, alpha-chain CDR3, beta-chain V sequence, alpha-chain V a sequence, beta-chain J sequence, alpha-chain J sequence of a TCR.

The TCR sequences may be from antigen-naive and antigen-experienced (memory) TCR repertoire of the subject. The TCR sequences may be only from antigen-experienced (memory) TCR repertoire of the subject.

The TCR sequences may be from a TCR repertoire present in a whole-blood sample, from other tissues such as synovial fluid, or bone marrow. The TCR sequences may be from a TCR repertoire present in a peripheral blood mononuclear cell (PBMC) sample derived from blood. The TCR repertoire may originate from T-cells stained with a selection of markers (e.g. antibodies when using flow cytometry) that allow isolation, and subsequent TCR sequencing, of specific T-cell subsets (e.g. naive CD4+ T-cells or antigen-experienced CD8+ T-cells). Method for sequences at least a part of a TCR repertoire of a subject are known in the art, for instance, Bacher, P., & Scheffold, A. (2013). Flow-cytometric analysis of rare antigen-specific T cells. Cytometry Part A, 83(8), 692-701; Ogg, G. S., & McMichael, A. J. (1998). HLA-peptide tetrameric complexes. Current opinion in immunology, 10(4), 393-396; Vollers, S. S., & Stern, L. J. (2008). Class II major histocompatibility complex tetramer staining: progress, problems, and prospects. Immunology, 123(3), 305-313; Bacher, P., Schink, C., Teutschbein, J., Kniemeyer, O., Assenmacher, M., Brakhage, A. A., & Scheffold, A. (2013). Antigen-reactive T cell enrichment for direct, high-resolution analysis of the human naive and memory Th cell repertoire. The Journal of Immunology, 1202221; Benveniste, P. M., Roy, S., Nakatsugawa, M., Chen, E. L., Nguyen, L., Millar, D. G., . . . & Zúñiga-Pflücker, J. C. (2018). Generation and molecular recognition of melanoma-associated antigen-specific human yδ T cells. Science immunology, 3(30), eaav4036.

The predictive model is a mathematical model or transformation generated or trained using a dataset comprising TCR sequences known to bind specifically to at least one (preferably one) model epitope. The selected predictive model is queried by receiving as an input a TCR sequence. The selected predictive model produces an output indicative of a likelihood of specific binding by the TCR sequence to the query epitope. The output might be a direct output from the model, or an output that has been processed or transformed into a numerical likelihood of specific binding on a scale. The scale might have a first limit (no/low likelihood of specific binding) and a second limit (high likelihood of specific binding). The first limit might be lower than the second limit. Examples of first and second limits are 0-1, and 0-100. It is appreciated that the first and second limits can be adapted according to requirements. The numerical likelihood of specific binding may indicate a structural or sequence similarity to the query epitope, related to a specific binding by the TCR sequence to the model epitope.

In general a predictive model that is a machine learning models outputs a confidence value. A predictive model that uses a distance metrics provide an output based on the calculated distance. The distance may be the lowest value found when comparing to the dataset.

An indication of specific binding is a dissociation constant; given current knowledge, a dissociation constant of 10⁻⁸ M or better may considered specific binding.

The predictive model is queried multiple times, one for each TCR sequence in the sequence data.

A Responsiveness Score is determined for the query epitope from the outputs of the predictive model for sequence data, namely for each and every TCR sequence in the sequence data. A single Responsiveness Score is indicative of the immune responsiveness of the subject for the query epitope. The Responsiveness Score is a likelihood of immune responsiveness for the subject towards the query epitope. The Responsiveness Score is preferably a number on a scale that has a first limit (no/low likelihood of immune responsiveness) and a second limit (high likelihood of immune responsiveness). The first limit might be lower than the second limit. Examples of first and second limits are 0-1, and 0-100.

The skilled person may determine a Responsiveness Score using various techniques, that are influenced by various factors including one or more of the type of predictive model, quantity of TCR sequences in the sequence data, desired scale, the degree of similarity or matching between the query epitope and model epitope.

One way of determining the Responsiveness Score is from an output, optionally processed, of a machine leaning model that is a confidence score.

Another way of determining the Responsiveness Score comprises counting the number of TCR sequence in the sequence data with a likelihood of specific binding to the query epitope above a specific binding (SP) threshold.

The number of TCR sequences counted may be divided by the total number of TCR sequences in the sequence data to arrive at the Responsiveness Score.

The number of TCR sequences counted may be divided by the number of TCR sequences in the sequence data predicted to specifically bind to a different antigen that is not expected be immunogenic in the subject and is unrelated to the target of interest to arrive at the Responsiveness Score. The specific binding (SP) threshold is typically above a mid-point in the scale (e.g. above 50%)

Another way of determining the Responsiveness Score comprises calculating a Shannon entropy of (all the) TCR sequence in the sequence data with a likelihood of specific binding to the query epitope above the specific binding (SP) threshold. The calculation may account for a read count of a TCR sequence of T-cell clonotype.

The Shannon entropy may be calculated using formula:

Σfₗ log(fₗ) wherein

fₗ is a frequency a TCR sequence in the sequence data with a likelihood of specific binding to the query epitope above the SP threshold.

The Shannon entropy calculated may be divided by the total Shannon entropy of TCR sequences in the sequence data to arrive at the Responsiveness Score.

The Shannon entropy calculated may be divided by the Shannon entropy of TCR sequences in the sequence data predicted to specifically bind to a different antigen that is not expected be immunogenic in the subject and is unrelated to the target of interest to arrive at the Responsiveness Score.

Another way of determining the Responsiveness Score comprises calculating a Simpson's diversity of (all the) TCR sequence in the sequence data with a likelihood of specific binding to the query epitope above the specific binding (SP) threshold. The calculation may account for a read count of a TCR sequence of T-cell clonotype

The Simpson's diversity may be calculated using formula:

1−(Σcₗ(cₗ−1))/C(C−1) wherein

cₗ is a count of a TCR sequence in the sequence data with a likelihood of specific binding to the query epitope above the SP threshold, and
C is the total number of TCR sequences in the sequence data.

The Simpson's diversity calculated may be divided by the total Simpson's diversity of TCR sequences in the sequence data to arrive at the Responsiveness Score.

The Simpson's diversity calculated may be divided by the Simpson's diversity of TCR sequences in the sequence data predicted to specifically bind to a different antigen that is not expected be immunogenic in the subject and is unrelated to the target of interest to arrive at the Responsiveness Score.

The Responsiveness Score may be used to determine an efficacy of a vaccine for a subject or for a population, when the vaccine contains at least one query epitope. A population may be composed of one or more sub-populations.

The efficacy of a vaccine may be an indication of a likelihood of reduction of disease in a vaccinated subject or population compared with an unvaccinated subject or population. It may be an indication of elicitation of immune response. The efficacy of a vaccine may be a number on a scale that has a first limit (no/low likelihood of reduction) and a second limit (high likelihood of reduction). The first limit might be lower than the second limit. Examples of first and second limits are 0-1, and 0-100.

The efficacy of a vaccine may be a level. The number of levels may be any, for instance 3 to 10, preferably 4 to 8 levels. There may be 2, 4, 6, or 8 levels. The levels may divided be within the first and second limits of the numerical scale. The lowest level (e.g. 1^{st} level) may correlate with or contain the first limit, the highest level (e.g. 4^{th} level) may correlate with or contain the second limit. The levels may be determined according to a categorisation of Responsiveness Scores.

The efficacy of a vaccine is related to the Responsiveness Score for each and every query epitope in the vaccine.

The efficacy of the vaccine for the subject, wherein the vaccine contains one query epitope may be determined from the Responsiveness Scores for the query epitope. The efficacy of the vaccine for the subject, wherein the vaccine contains a plurality of query epitopes may be determined by averaging the query epitope Responsiveness Scores; it is an aspect that only query epitope Responsiveness Scores above a threshold level (e.g. 80%) are considered for averaging.

The efficacy of the vaccine for a population wherein the vaccine contains one query epitopes may be determined by averaging the query epitope Responsiveness Scores for each member of the population.

The efficacy of the vaccine for a population, wherein the vaccine contains a plurality of query epitopes may be determined by averaging the query epitope Responsiveness Scores for the vaccine to arrive at a vaccine average (VA) Responsiveness Score for each subject, then to average the VA Responsiveness Scores across the subjects of the population. It is an aspect that only query epitope Responsiveness Scores above a threshold level (e.g. 80%) are considered in determining the vaccine average (VA) Responsiveness Score.

The model may be a machine learning (ML) model also known as an artificial intelligence model. The model may be a trained ML model. An ML model learns common patterns present the training dataset. Machine learning models are widely known in the art, for instance, from James, G, Witten, D, Hastie, T, & Tibshirani, R (2013), An introduction to statistical learning (Vol. 112). New York: Springer. The ML model may be any, including but not limited to Random forest; Conditional random field; Bayesian network; Support vector machine; Neural network; Convolutional neural network; Recurrent neural network; General adversarial neural network; K-nearest neighbor model; position-specific weight matrix; enriched k-mers; association rules; decision trees; Hidden Markov Model, or variants thereof, to name some of the commonly-known ML models. A ML model typically outputs a confidence score.

The ML model may be trained using the dataset comprising TCR sequences known to bind specifically to the model epitope thereof that is known as a training dataset. The training dataset may additionally contain TCR sequences known not to bind specifically to the model epitope.

The ML model may take as a query input one or more features derived from an amino acid TCR sequence. A feature may be a position-specific amino acid Boolean vector, sequence-specific amino acid Boolean vector, position-specific numerical vector representing mutation probabilities between amino acids or a variant thereof (such as PAM of BLOSUM matrices), a position-specific numerical vector representing amino acid physicochemical properties, or sequence-specific numerical vector representing amino acid physicochemical properties, or variant thereof. The ML model may use a decision boundary within the feature space that separates from the training dataset TCR sequences known to bind specifically to the antigen or one or more epitopes thereof from TCR sequences known not to bind specifically to the antigen or one or more epitopes thereof.

The ML model may output a score or a set of scores for each TCR in the sequence data. A cut-off for sufficient confidence may be established using false discovery rate, false positive rate or precision estimations on independent trial data.

The predictive model may predict specific binding using a score based on a Levenshtein distance from a TCR sequence within sequence (subject) data to a closest sequence in the dataset. A score threshold of 0, 1 or 2 may be used.

The predictive model may predict specific binding using a score based on a Hamming distance from a TCR sequence within sequence (subject) data to a closest sequence in the dataset. A score threshold of 0, 1 or 2 could then be used.

The predictive model may predict specific binding using a score based on a sequence alignment score of a TCR sequence within sequence (subject) data to a closest sequence in the dataset.

The dataset is used to generate or train the predictive model. The dataset comprises TCR sequences known to bind specifically to the model epitope. An indication of specific binding is a dissociation constant; given current knowledge, a dissociation constant of 10⁻⁸ M or better may considered specific binding. Specific binding may be measured using techniques known in the art such as Bacher, P., & Scheffold, A. (2013). Flow-cytometric analysis of rare antigen-specific T cells. Cytometry Part A, 83(8), 692-701.

The dataset may further comprise TCR sequences known not to bind specifically to the model epitope.

A TCR sequence in the dataset may comprise one of TCR beta-chain, TCR alpha-chain, or both, or part thereof. A TCR sequence in the dataset may comprise one of a complementary determining region of a TCR beta-chain, TCR alpha-chain, or both, or part thereof. A TCR sequence in the dataset may comprise one beta-chain CDR3, alpha-chain CDR3, beta-chain V sequence, alpha-chain V a sequence, beta-chain J sequence, alpha-chain J sequence of a TCR. The dataset may contain one type of sequence per predictive model.

The dataset (152) may comprise:
TCR sequences (156) known to bind specifically to a (preferably one) model epitope,
the model epitope sequence,

The dataset (152) may comprise:
TCR sequences (156) known to bind specifically to one model epitope,
the model epitope sequence.

The dataset (152) may comprise:
two or more model epitope sequences,
TCR sequences (156) known to bind specifically to the two or more model epitopes sequences.

Typically a single model epitope may be 7 to 33 amino acids in length.

It is appreciated that, depending on the type, the single predictive model may be capable of predicting specific binding of one query epitope, or of predicting specific binding more than one query epitope e.g. 2 or 3 epitopes.

A population may be composed of one or more sub-populations. A population may be of mixed sub-populations (i.e. several different sub-populations) or of one type of sub-population. At least one or all predictive models and hence datasets may be derived from a mixed sub-population of subjects. At least one or all predictive models and hence datasets may be derived from a single sub-population of subjects. Accordingly, the methods described herein may be applied to mixed subpopulations and/or to one specific sub-population of subjects. A reference set of subjects may be used, representative of the population or sub-population. A sub-population may be based on gender, age, ethnicity. A sub-population may be based on MHC genotype, optionally where members of the same sub-population have the same HLA allele for a given HLA locus.

Where an efficacy of a vaccine, or other type of prediction is made in respect of a substance or vaccine for a population, the Responsive Scores are determined from the TCR sequences for each representative subject of the population. Where the vaccine or substance contains multiple query epitopes, the Responsive Scores may be first consolidated (e.g. averaged) for the substance or vaccine, then further consolidated (e.g. averaged) for the population or set of representative subjects.

The method may be used for obtaining at least one Responsiveness Score for a vaccine in a subject to prior administration, wherein
the sequence data comprises TCR sequences of part of the TCR repertoire of the subject prior to vaccine administration,
at least one query epitope is identified from the vaccine that sequence matches a model epitope in the model database and the predictive model (used in the querying step) is linked to the matched model epitope in the model database,
a Responsiveness Score is obtained for each query epitope of the vaccine.

The method may be used for obtaining at least one Responsiveness Score for a vaccine in a population to prior administration, wherein
the sequence data comprises TCR sequences of part of the TCR repertoire of each subject of a set of reference subjects representative of the population prior to vaccine administration,
at least one query epitope is identified from the vaccine that sequence matches a model epitope in the model database and the predictive model (used in the querying step) is linked to the matched model epitope in the model database,
a Responsiveness Score is obtained for each query epitope of the vaccine for each reference subject.

The Responsiveness Score for each query epitope may be averaged across the set of reference subjects to obtain a population Responsiveness Score for each query epitope. The population may be made of up at least one sub-population of subjects. Where the population is made up of one sub-population, the predictive model may have been trained or generated using a dataset derived from the same single sub-population of subjects.

The method may be used for evaluating an efficacy of a vaccine in a subject to prior administration, wherein
the sequence data comprises TCR sequences of part of the TCR repertoire of the subject prior to vaccine administration,
at least one query epitope is identified from the vaccine that sequence matches a model epitope in the model database and the predictive model (used in the querying step) is linked to the matched model epitope in the model database,
the efficacy of the vaccine for the subject is determined from the Responsiveness Score of each query epitope.

More specifically, provided herein is a method for evaluating efficacy of a vaccine (170) in a subject by determining an immune responsiveness to at least one query epitope (126) identified from the vaccine (170) comprising:

receiving sequence data (122) comprising TCR sequences of at least a part of a TCR repertoire of a subject prior to vaccine administration,
selecting, for each query epitope (126), a predictive model (160) from a plurality of predictive models (PM_{ME-A}, PM_{ME-B}, PM_{ME-C}, . . . ),
   wherein each predictive model (PM_{ME-A}, PM_{ME-B}, PM_{ME-C}, . . . ) in the plurality of predictive models has been generated or trained using a dataset comprising a plurality of TCR sequences known to bind specifically to a model epitope (ME-A, ME-B, ME-C, . . . ),
   said predictive model (160) selected according to a sequence identity match between the model epitope (ME-A, ME-B, ME-C, . . . ) and query epitope (126),
      querying (130) each selected predictive model (160) with the sequence data (122),
determining (140) from outputs of the selected predictive model (160) a Responsiveness Score for each query epitope (126) indicative of the immune responsiveness of the subject to the query epitope (126),
evaluating (140) from the Responsiveness Score of each query epitope (126) the efficacy of the vaccine for the subject.

The sequence identity match between the model epitope (ME-A, ME-B, ME-C, . . . ) and query epitope (126) may be at least 80% or 90%. The number of query epitopes (126) in a set (120) may be at least 1, 2, or 3. The efficacy of the vaccine may be based on potential of T-cell activation.

The vaccine efficacy for the subject may be determined by ranking the query epitopes according to Responsiveness Score. Query epitopes (126) with a higher Responsiveness Score is indicative of a higher immune responsiveness. The highest ranking query epitope or epitopes (e.g. top 50%, 40% or 10%) may be considered for efficacy evaluation e.g. by comparison with a threshold Responsiveness Score. The query epitope or epitopes with a Responsiveness Score above a threshold value (e.g. 50%, 40% or 10%) may be considered efficacious.

The method may be used for evaluating efficacy of a vaccine in a population to prior administration, wherein
the sequence data comprises TCR sequences of part of the TCR repertoire of each subject of a set of reference subjects representative of the population prior to vaccine administration,
at least one query epitope is identified from the vaccine that sequence matches a model epitope in the model database and the predictive model (used in the querying step) is linked to the matched model epitope in the model database,
the efficacy of the vaccine for the set of reference subjects is determined from the Responsiveness Scores for each query epitope for the set of reference subjects.

More specifically, provided herein is a method for evaluating efficacy of a vaccine (170) in a population by determining an immune responsiveness to at least one query epitope (126) identified from the vaccine (170) comprising:

receiving sequence data (122) comprising TCR sequences of at least a part of a TCR repertoire of each subject of a set of reference subjects prior to vaccine administration,
selecting, for each query epitope (126) in the set of query epitopes (120), a predictive model (160) from a plurality of predictive models (PM_{ME-A}, PM_{ME-B}, PM_{ME-C}, . . . ),
   wherein each predictive model (PM_{ME-A}, PM_{ME-B}, PM_{ME-C}, . . . ) in the plurality of predictive models has been generated or trained using a dataset comprising a plurality of TCR sequences known to bind specifically to a model epitope (ME-A, ME-B, ME-C, . . . ),
   said predictive model (160) selected according to a sequence identity match between the model epitope (ME-A, ME-B, ME-C, . . . ) and query epitope (126),
querying (130) each selected predictive model (160) with the sequence data (122),
determining (140) from outputs of the selected predictive model (160) a Responsiveness Score for each query epitope (126) indicative of the immune responsiveness of the subject to the query epitope (126)
determining (140) from the Responsiveness Scores the efficacy of the vaccine for the set of reference subjects.

Responsiveness Scores for each query epitope (126) in set (120) of query epitopes (126) may be determined for each subject of the set of reference subjects. The efficacy of the vaccine for the population may be determined from the Responsiveness Scores of the set of the reference subjects. The set of reference subject may be indicative for the population. The sequence identity match between the model epitope (ME-A, ME-B, ME-C, . . . ) and query epitope (126) may be at least 80% or 90%. The number of query epitopes (126) in the vaccine may be at least 1, 2, or 3. The efficacy of the vaccine may be based on potential of T-cell activation.

The efficacy may be determined for a population that is made of up at least one sub-population of subjects. Where the population is made up of one sub-population, the predictive model may have been trained or generated using a dataset derived from the same single sub-population of subjects.

The efficacy of the vaccine may be determined from a ranking of the query epitopes (126) by Responsiveness Scores. A measure of the efficacy of the vaccine for the population may be a frequency of reference subjects having a responsiveness score above a threshold value. According to one example, the determination of vaccine efficacy for a population may be based on median Responsiveness Scores. For each query epitope (126), the Responsiveness Scores for the reference subjects may sorted from high to low. The median may then be defined as the Responsiveness Score at the N/2 position of the ranked Responsiveness Scores, where N is the number of reference subjects. The median Responsiveness Score may then be compared across all the Query epitopes (126), and the top ranking Query epitopes with the highest median (e.g. top 1, 2, 3 or more) are considered for evaluation e.g. by comparison with a threshold Responsiveness Score. The query epitope or epitopes with a Responsiveness Score above a threshold value (e.g. 50%, 40% or 10%) may be considered efficacious.

The method may be used for predicting for a subject an optimal vaccine from a set of query epitopes prior to administration, wherein:
the sequence data comprises TCR sequences of part of the TCR repertoire of each subject prior to vaccine administration,
each query epitope is searched against the model database to find a match to a model epitope and the predictive model (used in the querying step) is linked to the matched model epitope in the model database,
a Responsiveness Score is obtained for each query epitope,
the optimal vaccine for the subject is determined from the Responsiveness Score of each query epitope.

More specifically, provided herein is a method (100) for predicting for a subject an optimal vaccine composition from a set (120) of query epitopes (126), by determining an immune responsiveness of the subject to each query epitope (126) in the set (120) comprising:
receiving sequence data (122) comprising TCR sequences of at least a part of a TCR repertoire of the subject prior to vaccine administration,
selecting, for each query epitope (126) in the set of query epitopes (120), a predictive model (160) from a plurality of predictive models (PM_{ME-A}, PM_{ME-B}, PM_{ME-C}, . . . ),
   wherein each predictive model (PM_{ME-A}, PM_{ME-B}, PM_{ME-C}, . . . ) in the plurality of predictive models has been generated or trained using a dataset comprising a plurality of TCR sequences known to bind specifically to one model epitope (ME-A, ME-B, ME-C, . . . ),
   said predictive model (160) selected according to a sequence identity match between the model epitope (ME-A, ME-B, ME-C, . . . ) and query epitope (126),
querying (130) each selected predictive model (160) with the sequence data (122),
determining (140) from outputs of the selected predictive model (160) a Responsiveness Score for each query epitope (126) in the set (120) indicative of the immune responsiveness of the subject to the query epitope (126),
predicting the optimal vaccine composition for the subject from the Responsiveness Scores.

The sequence identity match between the model epitope (ME-A, ME-B, ME-C, . . . ) and query epitope (126) may be at least 80% or 90%. The number of query epitopes (126) in a set (120) may be at least 1, 2, or 3. The optimal vaccine may have the highest potential of T-cell activation.

The optimal vaccine for the subject may be determined by ranking the query epitopes according to Responsiveness Score. Query epitopes (126) with a higher Responsiveness Score is indicative of a higher immune responsiveness. The highest ranking query epitope or epitopes (e.g. top 50%, 40% or 10%) may be used in the optimal vaccine. The optimal vaccine for the subject may be determined by using a threshold Responsiveness Score.

The query epitope or epitopes with a Responsiveness Score above a threshold value (e.g. 50%, 40% or 10%) may be used in the optimal vaccine.

The method may be used for predicting for a population an optimal vaccine from a set of query epitopes prior to administration, wherein:
the sequence data comprises TCR sequences of part of the TCR repertoire of each subject of a set of reference subjects prior to vaccine administration,
each query epitope is searched against the model database to find a match to a model epitope and the predictive model (used in the querying step) is linked to the matched model epitope in the model database,
a set of Responsiveness Scores is obtained for each subject of the set, the set containing a Responsiveness Score for each query epitope,
the optimal vaccine for the population is determined from the Responsiveness Score of each query epitope for the set of reference subjects.

More specifically, provided herein is a method (100) predicting for a population an optimal vaccine composition from a set (120) of query epitopes (126), by determining an immune responsiveness of each subject of a set of reference subjects to each query epitope (126) in the set (120) comprising:
receiving sequence data (122) comprising TCR sequences of at least a part of a TCR repertoire of each subject in the set of reference subjects prior to vaccine administration,
selecting, for each query epitope (126) in the set of query epitopes (120), a predictive model (160) from a plurality of predictive models (PM_{ME-A}, PM_{ME-B}, PM_{ME-C}, . . . ),
   wherein each predictive model (PM_{ME-A}, PM_{ME-B}, PM_{ME-C}, . . . ) in the plurality of predictive models has been generated or trained using a dataset comprising a plurality of TCR sequences known to bind specifically to a model epitope (ME-A, ME-B, ME-C, . . . ),
   said predictive model (160) selected according to a sequence identity match between the model epitope (ME-A, ME-B, ME-C, . . . ) and query epitope (126),
querying (130) the each selected predictive model (160) with the sequence data (122),
determining (140) from outputs of the selected predictive model (160) a Responsiveness Score for each query epitope (126) in the set (120) indicative of the immune responsiveness of the subject to the query epitope (126),
predicting the optimal vaccine composition for the population from the Responsiveness Scores for the set of reference subjects.

Responsiveness Scores for each query epitope (126) in set (120) of query epitopes (126) may be determined for each subject of the set of reference subjects. The optimal vaccine composition for the set of reference subjects may be predicted from Responsiveness Scores for the set of reference subjects. The sequence identity match between the model epitope (ME-A, ME-B, ME-C, . . . ) and query epitope (126) may be at least 80% or 90%. The number of query epitopes (126) in a set (120) may be at least 1, 2, or 3. The optimal vaccine has the highest potential of T-cell activation.

The set of reference subjects is representative of the population. The prediction may be determined for different sub-populations of subjects according to the composition of the set of reference subjects.

The Responsiveness Score for each query epitope may be averaged across the set of reference subjects to obtain a population Responsiveness Score for each query epitope. The optimal vaccine for the population may be determined by ranking the query epitopes according to individual or population Responsiveness Scores. The highest ranking query epitope or epitopes (e.g. top 50%, 40% or 10%) may be used in the optimal vaccine. According to one example, the determination of an optimum vaccine for a population may be based on median Responsiveness Scores. For each query epitope (126), the Responsiveness Scores for the reference subjects may sorted from high to low. The median may then be defined as the Responsiveness Score at the N/2 position of the ranked Responsiveness Scores, where N is the number of reference subjects. The median Responsiveness Score may then be compared across all the Query epitopes (126), and the top ranking Query epitopes with the highest median (e.g. top 1, 2, 3 or more) may indicated for use in optimal vaccine composition.

The optimal vaccine for the subject may be determined by using a threshold Responsiveness Score. The query epitope or epitopes above a threshold value (e.g. 50%, 40% or 10%) may be used in the optimal vaccine.

The method may be used for predicting unresponsiveness in a subject to a vaccine, wherein
the sequence data comprises TCR sequences of part of the TCR repertoire of each subject of the subject prior to vaccine administration,
at least one query epitope is identified from the vaccine that sequence matches a model epitope in the model database and the predictive model (used in the querying step) is linked to the matched model epitope in the model database,
a Responsiveness Scores is obtained for each query epitope,
the unresponsiveness of the vaccine for subject is determined from the Responsiveness Scores for each query epitope.

A measure of the unresponsiveness of the vaccine for the subject may be a Responsiveness Score for all the query epitopes below a threshold value.

The method may be used for predicting unresponsiveness in a population to a vaccine, wherein
the sequence data comprises TCR sequences of part of the TCR repertoire of each subject of a set of reference subjects prior to vaccine administration,
at least one query epitope is identified from the vaccine that sequence matches a model epitope in the model database and the predictive model (used in the querying step) is linked to the matched model epitope in the model database,
a set of Responsiveness Scores is obtained for each subject of the set, the set containing a Responsiveness Score for each query epitope,
the unresponsiveness of the vaccine for the set of reference subjects is determined from the responsiveness scores for the set of reference subjects.

The set of reference subjects is representative of the population.

A measure of the unresponsiveness of the vaccine for the set of reference subjects may be a frequency of reference subjects having a Responsiveness Score below a threshold value. The unresponsiveness may be determined for different sub-populations of subjects according to the composition of the set of reference subjects. The query epitopes may be from a same target protein or different target proteins.

A measure of the unresponsiveness of the vaccine for the set of reference subjects may be determined from

a. an average of the Responsiveness Scores for each query epitope across each reference subject of the set
b. an average of the averaged Responsiveness Scores of a.

The method may be used for determining timing for a booster dose of a vaccine in a subject after administration of a prime dose same vaccine to the subject, wherein
at least one query epitope is identified from the vaccine that sequence matches a model epitope in the model database and the predictive model (used in the querying step) is linked to the matched model epitope in the model database,
the sequence data comprising TCR sequences of part of the TCR repertoire of the subject is received at one or more time intervals after administration of the vaccine prime dose and the Responsiveness Score for each query epitope is determined at each time interval,
the Responsiveness Scores at the different time intervals are used to determine the timing for the booster dose.

The Responsive Score below a threshold value may indicate a need for administration of the booster dose of a vaccine. The query epitopes may be from a same target protein or different target proteins.

The method may be used for determining monitoring post-administration efficacy of a vaccine in a subject after administration of the same vaccine to the subject, wherein
at least one query epitope is identified from the vaccine that sequence matches a model epitope in the model database and the predictive model (used in the querying step) is linked to the matched model epitope in the model database,
the sequence data comprising TCR sequences of part of the TCR repertoire of the subject is received at one (pref. two) or more time intervals after and optionally one or more time intervals before administration of the vaccine and the Responsiveness Score determined at each time interval,
the post-administration efficacy of the vaccine is determined from the Responsiveness Scores for each query epitope.

The post-administration efficacy of the vaccine may determined from a change (pref. increase) in the responsiveness scores between the different time intervals. The query epitopes may be from a same target protein or different target proteins.

The method may be used for determining monitoring post-administration efficacy of a vaccine in a population after administration of the same vaccine to the subject, wherein
the sequence data comprises TCR sequences of part of the TCR repertoire of each subject of a set of reference subjects after vaccine administration, and at one (pref. two) or more time intervals after and optionally one or more time intervals before vaccine administration
at least one query epitope is identified from the vaccine that sequence matches a model epitope in the model database and the predictive model (used in the querying step) is linked to the matched model epitope in the model database,
a Responsiveness Score is determined at each time interval,
the post-administration efficacy of the vaccine for the population is determined from Responsiveness Scores for each query epitope for the set of reference subjects.

The set of reference subjects is representative of the population. The efficacy may be determined for different sub-populations of subjects according to the composition of the set of reference subjects. The query epitopes may be from a same target protein or different target proteins. The post-administration efficacy of the vaccine may be determined from a change (pref. increase) in the responsiveness scores between the different time intervals.

The method may be used for determining presence or status of an infectious disease in a subject, wherein
at least one query epitope is identified from the infectious disease that sequence matches a model epitope in the model database and the predictive model (used in the querying step) is linked to the matched model epitope in the model database,
the sequence data comprises TCR sequences of part of the TCR repertoire of the subject suspected to have or already having the infectious disease,
the presence or status of the infectious disease is determined from the Responsiveness Scores for each query epitope.

The presence or status of the infectious disease is determined by using a threshold Responsiveness Score. The query epitope or epitopes with a Responsiveness Score above a threshold value (e.g. 50%, 60% or 90%) may indicate a presence or status of the infectious disease.

The method may be a computer implemented method.

The method may be performed on a computing device or system.

Provided is a computing device or system configured for performing the method described herein.

Provided is a computer program or computer program product having instructions which when executed by a computing device or system cause the computing device or system to perform the method described herein.

Provided is a computer readable medium having stored thereon instructions which when executed by a computing device or system cause the computing device or system to perform (each of the steps of) the method described herein.

Provided is a data stream which is representative of a computer program or computer program product described herein.

### Example

A total of 34 healthy volunteers (20-29 y: 10, 30-39 y: 7, 40-49 y: 16, 50+y: 1) without a history of hepatitis B virus (HBV) infection or previous HBV vaccination were included in this study after written informed consent. All volunteers received a diary to log their medication intake or episodes of illness, as these factors could influence the general immune system and the immune response upon vaccination. In this study, each volunteer received a Hepatitis B surface Antigen (HBsAg) vaccine dose (Engerix-B, GSK) at days 0 and 30. The vaccination schedule was further completed at day 365. At days 0 and 60, serum was taken for anti-HBsAg titration. Individuals with anti-HBsAg levels below 10 IU/L were classified as non-responders whereas individuals with anti-HBsAg levels above 10 IU/L were considered as responders.

Peripheral blood mononuclear cells (PBMCs) were isolated from each volunteer, before vaccination and on days 60, 180 and 365 after vaccination, and frozen following standard operating procedures as detailed elsewhere (Ogunjimi, B. et al. Sci. Rep. 7, 1077 (2017)). After thawing and washing cryopreserved PBMCs, total CD4+ T cells were isolated by positive selection using CD4 magnetic microbeads (Miltenyi Biotech). Memory CD4+ T cells were sorted after gating on single viable CD3+CD4+CD8-CD45RO+ cells. The following fluorochrome-labeled monoclonal antibodies were used for staining: CD3-PerCP (BW264/56) (from Miltenyi Biotech), CD4-APC (RPA-T4) and CD45RO-PE (UCHT1) (both from BD Biosciences) and CD8-Pacific Orange (3B5) (from Thermo Fisher Scientic). Cells were stained at room temperature for 20 minutes and sorted with FACSAria II (BD Biosciences). Sytox blue (Thermo Fisher Scientic) was used to exclude non-viable cells. TCRβ DNA from memory CD4+ T cells was sequenced using Adaptive Biotechnologies' ImmunoSEQ hsTCRβ kit on an Illumina Miseq sequencer according to the manufacturer's protocol.

A model database containing a plurality of trained predictive models each linked with a model epitope was created using a TCR dataset created by stimulating PBMC with 15aa long peptides (JPT) derived from the HBsAg sequence as present in the vaccine (Engerix-B, GSK). Epitope-specific T-cells were sorted using a carboxyfluorescein succinimidyl ester (CFSE) proliferation assay, after which RNA extraction occurred. The QIAGEN TCR kit was used for TCR sequencing of epitope-specific T-cells.

The model epitopes in the model database were used to search the HBsAg vaccine for putative query epitopes with 100% (i.e. ≥80% or ≥90%) sequence identity.

The HBsAg vaccine (Engerix-B, GSK) was found to contain 35 individual query epitopes according to Table 1.

TABLE 1
Sequences of 35 individual query epitopes of
the HBsAg vaccine (Engerix-B, GSK).

| | | |
|---|---|---|
| MENITSGFLGPLLVL | CKTCTTPAQGNSMFP | TVWLSAIWMMWYWGP |
| SEQ ID NO: 1 | SEQ ID NO: 14 | SEQ ID NO: 27 |
| TSLNFLGGSPVCLGQ | QGNSMFPSCCCTKPT | SAIWMMWYWGPSLYS |
| SEQ ID NO: 2 | SEQ ID NO: 15 | SEQ ID NO: 28 |
| SPVCLGQNSQSPTSN | KPTDGNCTCIPIPSS | AGFFLLTRILTIPQS |
| SEQ ID NO: 3 | SEQ ID NO: 16 | SEQ ID NO: 29 |
| LGQNSQSPTSNHSPT | GNCTCIPIPSSWAFA | LYSIVSPFIPLLPIF |
| SEQ ID NO: 4 | SEQ ID NO: 17 | SEQ ID NO: 30 |
| SPTSCPPICPGYRWM | CIPIPSSWAFAKYLW | VSPFIPLLPIFFCLW |
| SEQ ID NO: 5 | SEQ ID NO: 18 | SEQ ID NO: 31 |
| CPPICPGYRWMCLRR | PSSWAFAKYLWEWAS | IPLLPIFFCLWVYI |
| SEQ ID NO: 6 | SEQ ID NO: 19 | SEQ ID NO: 32 |
| TSGFLGPLLVLQAGF | LVLQAGFFLLTRILT | LLTRILTIPQSLDSW |
| SEQ ID NO: 7 | SEQ ID NO: 20 | SEQ ID NO: 33 |
| IIFLFILLLCLIFLL | AFAKYLWEWASVRFS | ILTIPQSLDSWWTS |
| SEQ ID NO: 8 | SEQ ID NO: 21 | SEQ ID NO: 34 |
| FILLLCLIFLLVLLD | YLWEWASVRFSWLSL | PQSLDSWWTSLNFLG |
| SEQ ID NO: 9 | SEQ ID NO: 22 | SEQ ID NO: 35 |
| LCLIFLLVLLDYQGM | WASVRFSWLSLLVPF | |
| SEQ ID NO: 10 | SEQ ID NO: 23 | |
| FLLVLLDYQGMLPVC | VPFVQWFVGLSPTVW | |
| SEQ ID NO: 11 | SEQ ID NO: 24 | |
| LLDYQGMLPVCPLIP | QWFVGLSPTVWLSAI | |
| SEQ ID NO: 12 | SEQ ID NO: 25 | |
| LIPGSTTTNTGPCKT | GLSPTVWLSAIWMMW | |
| SEQ ID NO: 13 | SEQ ID NO: 26 | |

Each predictive model linked to a model epitope matching a query epitope was queried with the total CD4+ memory TCR repertoire from each volunteer by applying a Hamming distance calculation between the distance of each TCR beta-chain CDR3 region amino acid sequence in the volunteer repertoire and each TCR beta-chain CDR3 region amino acid sequence in the model database for the epitopes.

A Responsiveness Score for each volunteer to the HBsAg vaccine was determined, by counting the number of TCRs in their sequenced TCR repertoires with a calculated distance of 0. The count was then normalized by the amount of TCR sequences contained in the database for all epitopes. The final normalized count was then used as the Responsiveness Score for each volunteer.

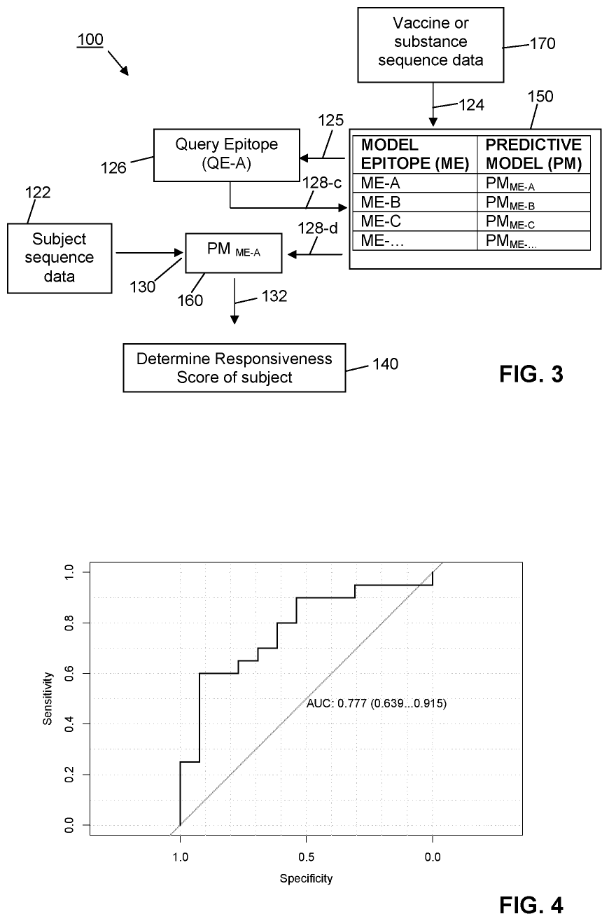

An ROC curve was generated comparing the individual Responsiveness Scores to the results obtained from the anti-HBVs titration (FIG. 4).

There was a good correlation between Responsiveness Score (prediction) and anti-HBVs titration (actual) data, with an AUC (area under the curve) of 0.78. The Responsiveness Score calculated prior to vaccination is hence a good indicator of the vaccine-induced antibody response 30 days after a second Engerix vaccine.

While T-cells are known to assist in the activation of B-cells in producing antibodies following vaccination, correlations between T-cells and antibodies following vaccination cannot always be found. The results show that T-cells that are already present in the memory CD4+ T-cell repertoire prior to the vaccination event are of key importance in the development of antibodies following vaccination. This finding was currently unknown. The outlined technology allows identification of these reactive T-cells in a high-throughput manner and quantification into a single metric to predict vaccine response (in this example antibody response following vaccination).

1. A method (100) for predicting for a subject an optimal vaccine composition from a set (120) of query epitopes (126), by determining an immune responsiveness of the subject to each query epitope (126) in the set (120) comprising:
receiving sequence data (122) comprising TCR sequences of at least a part of a TCR repertoire of the subject prior to vaccine administration,
selecting, for each query epitope (126) in the set of query epitopes (120), a predictive model (160) from a plurality of predictive models (PM_{ME-A}, PM_{ME-B}, PM_{ME-C}, . . . ),
wherein each predictive model (PM_{ME-A}, PM_{ME-B}, PM_{ME-C}, . . . ) in the plurality of predictive models has been generated or trained using a dataset comprising a plurality of TCR sequences known to bind specifically to one model epitope (ME-A, ME-B, ME-C, . . . ),
said predictive model (160) selected according to a sequence identity match between the model epitope (ME-A, ME-B, ME-C, . . . ) and query epitope (126),
querying (130) each selected predictive model (160) with the sequence data (122),
determining (140) from outputs of the selected predictive model (160) a Responsiveness Score for each query epitope (126) in the set (120) indicative of the immune responsiveness of the subject to the query epitope (126),
predicting the optimal vaccine composition for the subject from the Responsiveness Scores.

2. A method (100) for optimal vaccine composition from a set (120) of query epitopes (126), from a set (120) of query epitopes (126), by determining an immune responsiveness of each subject of a set of reference subjects to each query epitope (126) in the set (120) comprising:
receiving sequence data (122) comprising TCR sequences of at least a part of a TCR repertoire of each subject in the set of reference subjects prior to vaccine administration,
selecting, for each query epitope (126) in the set of query epitopes (120), a predictive model (160) from a plurality of predictive models (PM_{ME-A}, PM_{ME-B}, PM_{ME-C}, . . . ),
wherein each predictive model (PM_{ME-A}, PM_{ME-B}, PM_{ME-C}, . . . ) in the plurality of predictive models has been generated or trained using a dataset comprising a plurality of TCR sequences known to bind specifically to a model epitope (ME-A, ME-B, ME-C, . . . ),
said predictive model (160) selected according to a sequence identity match between the model epitope (ME-A, ME-B, ME-C, . . . ) and query epitope (126),
querying (130) each selected predictive model (160) with the sequence data (122),
determining (140) from outputs of the selected predictive model (160) a Responsiveness Score for each query epitope (126) in the set (120) indicative of the immune responsiveness of the subject to the query epitope (126),
predicting the optimal vaccine composition for the population from the Responsiveness Scores for the set of reference subjects.

3. A method for evaluating efficacy of a vaccine (170) in a subject by determining an immune responsiveness to at least one query epitope (126) identified from the vaccine (170) comprising:
receiving sequence data (122) comprising TCR sequences of at least a part of a TCR repertoire of a subject prior to vaccine administration,
selecting, for each query epitope (126), a predictive model (160) from a plurality of predictive models (PM_{ME-A}, PM_{ME-B}, PM_{ME-C}, . . . ),
wherein each predictive model (PM_{ME-A}, PM_{ME-B}, PM_{ME-C}, . . . ) in the plurality of predictive models has been generated or trained using a dataset comprising a plurality of TCR sequences known to bind specifically to a model epitope (ME-A, ME-B, ME-C, . . . ),
said predictive model (160) selected according to a sequence identity match between the model epitope (ME-A, ME-B, ME-C, . . . ) and query epitope (126),
querying (130) each selected predictive model (160) with the sequence data (122),
determining (140) from outputs of the selected predictive model (160) a Responsiveness Score for each query epitope (126) indicative of the immune responsiveness of the subject to the query epitope (126),
evaluating (140) from the Responsiveness Score of each query epitope (126) the efficacy of the vaccine for the subject.

4. A method for evaluating efficacy of a vaccine (170) in a population by determining an immune responsiveness to at least one query epitope (126) identified from the vaccine (170) comprising:
receiving sequence data (122) comprising TCR sequences of at least a part of a TCR repertoire of each subject of a set of reference subjects prior to vaccine administration,
selecting, for each query epitope (126) in the set of query epitopes (120), a predictive model (160) from a plurality of predictive models (PM_{ME-A}, PM_{ME-B}, PM_{ME-C}, . . . ),
wherein each predictive model (PM_{ME-A}, PM_{ME-B}, PM_{ME-C}, . . . ) in the plurality of predictive models has been generated or trained using a dataset comprising a plurality of TCR sequences known to bind specifically to a model epitope (ME-A, ME-B, ME-C, . . . ),
said predictive model (160) selected according to a sequence identity match between the model epitope (ME-A, ME-B, ME-C, . . . ) and query epitope (126),
querying (130) each selected predictive model (160) with the sequence data (122),
determining (140) from outputs of the selected predictive model (160) a Responsiveness Score for each query epitope (126) indicative of the immune responsiveness of the subject to the query epitope (126)
determining (140) from the Responsiveness Scores the efficacy of the vaccine for the set of reference subjects.

5. The method according claim 1, wherein the predictive model is a machine learning model trained using the training dataset.

6. The method according to claim 1, wherein the wherein the total quantity of TCR sequences in the sequence data (122) is a fraction of the total number of available TCR sequences in the repertoire of the subject.

7. The method according to claim 1, wherein sequence data (122) comprises TCR sequences that are only antigen-experienced TCR sequences from the TCR repertoire of the subject.

8. The method according to claim 1, wherein the vaccine comprises one or more of:
at least one amino acid chain (protein, polypeptide, peptide)
at least one nucleic acid (double or single stranded RNA, DNA; DNA-RNA hybrid)
at least one immune system cell (e.g. antigen presenting cell, T-cell, B-cell, macrophage),
at least one an infectious agent (e.g. prokaryotic cell (bacteria),
at least one eukaryotic cell (yeast),
at least one virus,
at least one prion,
and the at least one query epitope is present in the substance or vaccine as part of an amino acid sequence of the same length as the query epitope or longer than the query epitope, and/or as nucleic acid encoding said amino acid sequence of the same length as the query epitope or longer than the query epitope.


SEQUENCE LISTING (35 sequences)

SEQ ID NO: 1 - protein, 15 aa, Hepatitis B virus
  MISC_FEATURE (1)..(15): surface Antigens
MENITSGFLGPLLVL

SEQ ID NO: 2 - protein, 15 aa, Hepatitis B virus
  MISC_FEATURE (1)..(15): surface Antigens
TSLNFLGGSPVCLGQ

SEQ ID NO: 3 - protein, 15 aa, Hepatitis B virus
  MISC_FEATURE (1)..(15): surface Antigens
SPVCLGQNSQSPTSN

SEQ ID NO: 4 - protein, 15 aa, Hepatitis B virus
  MISC_FEATURE (1)..(15): surface Antigens
LGQNSQSPTSNHSPT

SEQ ID NO: 5 - protein, 15 aa, Hepatitis B virus
  MISC_FEATURE (1)..(15): surface Antigens
SPTSCPPICPGYRWM

SEQ ID NO: 6 - protein, 15 aa, Hepatitis B virus
  MISC_FEATURE (1)..(15): surface Antigens
CPPICPGYRWMCLRR

SEQ ID NO: 7 - protein, 15 aa, Hepatitis B virus
  MISC_FEATURE (1)..(15): surface Antigens
TSGFLGPLLVLQAGF

SEQ ID NO: 8 - protein, 15 aa, Hepatitis B virus
  MISC_FEATURE (1)..(15): surface Antigens
IIFLFILLLCLIFLL

SEQ ID NO: 9 - protein, 15 aa, Hepatitis B virus
  MISC_FEATURE (1)..(15): surface Antigens
FILLLCLIFLLVLLD

SEQ ID NO: 10 - protein, 15 aa, Hepatitis B virus
  MISC_FEATURE (1)..(15): surface Antigens
LCLIFLLVLLDYQGM

SEQ ID NO: 11 - protein, 15 aa, Hepatitis B virus
  MISC_FEATURE (1)..(15): surface Antigens
FLLVLLDYQGMLPVC

SEQ ID NO: 12 - protein, 15 aa, Hepatitis B virus
  MISC_FEATURE (1)..(15): surface Antigens
LLDYQGMLPVCPLIP

SEQ ID NO: 13 - protein, 15 aa, Hepatitis B virus
  MISC_FEATURE (1)..(15): surface Antigens
LIPGSTTTNTGPCKT

SEQ ID NO: 14 - protein, 15 aa, Hepatitis B virus
  MISC_FEATURE (1)..(15): surface Antigens
CKTCTTPAQGNSMFP

SEQ ID NO: 15 - protein, 15 aa, Hepatitis B virus
  MISC_FEATURE (1)..(15): surface Antigens
QGNSMFPSCCCTKPT

SEQ ID NO: 16 - protein, 15 aa, Hepatitis B virus
  MISC_FEATURE (1)..(15): surface Antigens
KPTDGNCTCIPIPSS

SEQ ID NO: 17 - protein, 15 aa, Hepatitis B virus
  MISC_FEATURE (1)..(15): surface Antigens
GNCTCIPIPSSWAFA

SEQ ID NO: 18 - protein, 15 aa, Hepatitis B virus
  MISC_FEATURE (1)..(15): surface Antigens
CIPIPSSWAFAKYLW

SEQ ID NO: 19 - protein, 15 aa, Hepatitis B virus
  MISC_FEATURE (1)..(15): surface Antigens
PSSWAFAKYLWEWAS

SEQ ID NO: 20 - protein, 15 aa, Hepatitis B virus
  MISC_FEATURE (1)..(15): surface Antigens
LVLQAGFFLLTRILT

SEQ ID NO: 21 - protein, 15 aa, Hepatitis B virus
  MISC_FEATURE (1)..(15): surface Antigens
AFAKYLWEWASVRFS

SEQ ID NO: 22 - protein, 15 aa, Hepatitis B virus
  MISC_FEATURE (1)..(15): surface Antigens
YLWEWASVRFSWLSL

SEQ ID NO: 23 - protein, 15 aa, Hepatitis B virus
  MISC_FEATURE (1)..(15): surface Antigens
WASVRFSWLSLLVPF

SEQ ID NO: 24 - protein, 15 aa, Hepatitis B virus
  MISC_FEATURE (1)..(15): surface Antigens
VPFVQWFVGLSPTVW

SEQ ID NO: 25 - protein, 15 aa, Hepatitis B virus
  MISC_FEATURE (1)..(15): surface Antigens
QWFVGLSPTVWLSAI

SEQ ID NO: 26 - protein, 15 aa, Hepatitis B virus
  MISC_FEATURE (1)..(15): surface Antigens
GLSPTVWLSAIWMMW

SEQ ID NO: 27 - protein, 15 aa, Hepatitis B virus
  MISC_FEATURE (1)..(15): surface Antigens
TVWLSAIWMMWYWGP

SEQ ID NO: 28 - protein, 15 aa, Hepatitis B virus
  MISC_FEATURE (1)..(15): surface Antigens
SAIWMMWYWGPSLYS

SEQ ID NO: 29 - protein, 15 aa, Hepatitis B virus
  MISC_FEATURE (1)..(15): surface Antigens
AGFFLLTRILTIPQS

SEQ ID NO: 30 - protein, 15 aa, Hepatitis B virus
  MISC_FEATURE (1)..(15): surface Antigens
LYSIVSPFIPLLPIF

SEQ ID NO: 31 - protein, 15 aa, Hepatitis B virus
  MISC_FEATURE (1)..(15): surface Antigens
VSPFIPLLPIFFCLW

SEQ ID NO: 32 - protein, 14 aa, Hepatitis B virus
  MISC_FEATURE (1)..(14): surface Antigens
IPLLPIFFCLWVYI

SEQ ID NO: 33 - protein, 15 aa, Hepatitis B virus
  MISC_FEATURE (1)..(15): surface Antigens
LLTRILTIPQSLDSW

SEQ ID NO: 34 - protein, 15 aa, Hepatitis B virus
  MISC_FEATURE (1)..(15): surface Antigens
ILTIPQSLDSWWTSL

SEQ ID NO: 35 - protein, 15 aa, Hepatitis B virus
  MISC_FEATURE (1)..(15): surface Antigens
PQSLDSWWTSLNFLG